(19) United States
(12) Patent Application Publication
(10) Pub. No.: US 2025/0161603 A1
(43) Pub. Date: May 22, 2025

(54) ELECTRONIC VAPOR PROVISION SYSTEM

(71) Applicant: NICOVENTURES TRADING LIMITED, London (GB)

(72) Inventor: Christopher LORD, London (GB)

(21) Appl. No.: 19/029,201

(22) Filed: Jan. 17, 2025

Related U.S. Application Data
(63) Continuation of application No. 18/592,585, filed on Mar. 1, 2024.
Application No. 18/592,585 is a continuation of application No. 17/517,607, filed on Nov. 2, 2021, now Pat. No. 11,931,507.
Application No. 17/517,607 is a continuation of application No. 16/677,113, filed on Nov. 7, 2019, now Pat. No. 11,185,649.
Application No. 16/677,113 is a continuation of application No. 14/401,511, filed on Nov. 14, 2014, now Pat. No. 10,477,893.

(30) Foreign Application Priority Data
May 14, 2012 (GB) 1208352.3

(51) Int. Cl. A61M 15/06; A24F 40/10; A24F 40/40; A24F 40/53; A24F 40/90; A61M 11/04; A61M 16/00

(52) U.S. Cl. CPC A61M 15/06; A24F 40/40; A24F 40/53; A24F 40/90; A61M 11/042; A24F 40/10; A61M 2016/0021; A61M 2016/0039; A61M 2205/3653; A61M 2205/8206

(57) ABSTRACT

An electronic vapor provision device includes a body and a vaporizer. The body includes a power cell and a processor, and the vaporizer is releasably connectable to the body. The processor is configured to enter a sleep mode when the vaporizer is connected to the body and the device is inactive for an inactive time. Furthermore, the processor is configured to leave the sleep mode and enter a usable mode when the vaporizer is disconnected from the body then reconnected to the body.

### PRIORITY CLAIM

This application is a continuation of application Ser. No. 18/592,585 filed Mar. 1, 2024, which is a continuation of application Ser. No. 17/517,607 filed Nov. 2, 2021, now U.S. Pat. No. 11,931,507, which is a continuation of application Ser. No. 16/677,113, filed Nov. 7, 2019, now U.S. Pat. No. 11,185,649, issued Nov. 30, 2021, which is a continuation of application Ser. No. 14/401,511, filed Nov. 14, 2014, now U.S. Pat. No. 10,477,893, issued Nov. 19, 2019, which in turn is a National Phase entry of PCT Application No. PCT/EP2013/059954, filed May 14, 2013, which claims priority from GB Patent Application No. 1208352.3, filed May 14, 2012, each of which is hereby fully incorporated herein by reference.

### FIELD

The specification relates to electronic vapor provision devices. More particularly, but not exclusively, the specification concerns electronic vapor provision devices in the form of electronic cigarettes.

### BACKGROUND

Electronic vapor provision devices are typically cigarette-sized and function by allowing a user to inhale a nicotine vapor from a liquid store by applying a suction force to a mouthpiece. Some electronic vapor provision devices have an airflow sensor that activates when a user applies the suction force and causes a heater coil to heat up and vaporize the liquid. Electronic vapor provision devices include electronic cigarettes.

### SUMMARY

In an embodiment there is provided an electronic vapor provision device comprising a body and a vaporizer, wherein the body comprises a power cell and a processor; the vaporizer is releasably connectable to the body; the processor is configured to enter a sleep mode when the vaporizer is connected to the body and the device is inactive for an inactive time; and the processor is configured to leave the sleep mode and enter a usable mode when the vaporizer is disconnected from the body then reconnected to the body.

This has the advantage that once the device has entered a sleep mode, it does not accidentally enter a normal mode. Entering a normal mode requires effort from the user.

The electronic vapor provision device may be an electronic cigarette.

The sleep mode can be a low power mode. Moreover, the sleep mode may be the lowest non-zero power mode of the device.

By remaining in a low power sleep mode the device remains active yet consumes very little power. This provides an efficient use of power and minimizes energy wastage.

The device has a further advantage that it can remain in a low power mode without the additional use of a switch to deactivate and activate.

Furthermore, the electronic vapor provision device can use less power in sleep mode than in usable mode.

Advantageously, the usable mode is a higher power state to enable a more rapid activation once the device is activated by a user.

The electronic vapor provision device may be inactive when not being used as a vapor provision device by a user.

The body may further comprise a capacitor; and the processor can be configured to first charge the capacitor and then detect whether the vaporizer is connected to the body by determining whether the capacitor is discharged.

The body may comprise first and second body connection terminals, and the vaporizer may comprise first and second vaporizer connection terminals. Furthermore, the device may be configured such that when the vaporizer is connected to the body, the first body connection terminal is connected to the first vaporizer connection terminal and the second body connection terminal is connected to the second vaporizer connection terminal.

The capacitor can be connected in parallel with the first and second body connection terminals. The computer can be configured to send out a pulse and the capacitor can be charged for a period of time equal to the width of the pulse. Moreover, the pulse may be a square wave.

In another embodiment there is provided the vaporizer of the electronic vapor provision device of other embodiments.

In another embodiment there is provided the an electronic vapor provision device body, comprising a power cell and a processor, and configured to be releasably connectable to a vaporizer; wherein the processor is configured to enter a sleep mode when the vaporizer is connected to the body and the device is inactive for an inactive time; and the processor is configured to leave the sleep mode and enter a usable mode when the vaporizer is disconnected from the body then reconnected to the body.

In another embodiment there is provided an electronic smoking device comprising a battery assembly and a vaporizer, where the battery assembly comprises a power cell and a computer, the vaporizer is releasably-attachable to the battery assembly and the computer comprises a computer processor, a memory and an input-output means; wherein when the vaporizer is attached to the battery assembly, the computer is configured in use to enter a sleep mode when the device is inactive for a predetermined inactive time.

In another embodiment there is provided a method of triggering a change in the mode of a processor of an electronic vapor provision device from a sleep mode to a usable mode, wherein the electronic vapor provision device comprises a body and a vaporizer; the body comprises a power cell and the processor; and the vaporizer is releasably connectable to the body; the method comprising disconnecting the vaporizer from the body and then reconnecting the vaporizer to the body.

As used herein, the term vapor includes an aerosol and other fluid streams for provision to a user by the electronic vapor provision device.

### BRIEF DESCRIPTION OF THE DRAWINGS

For a better understanding of the disclosure, and to show how example embodiments may be carried into effect, reference will now be made to the accompanying drawings in which:

FIG. 1 is a side perspective view of an electronic vapor provision device;

FIG. 2 is an exploded side perspective view of the electronic vapor provision device of FIG. 1;

FIG. 3 is a side sectional view through the device of FIG. 1;

FIG. 4 is a side perspective view of an electronic vapor provision device with separated mouthpiece and body;

FIG. 5 is a side perspective view of an electronic vapor provision device with separated mouthpiece, vaporizer and battery assembly;

FIG. 6 is a side sectional view through the electronic vapor provision device of FIG. 4 with connected mouthpiece and body;

FIG. 7 is a side sectional view of a battery assembly having a capacitor; and

FIG. 8 is a circuit diagram for the battery assembly of FIG. 7.

### DETAILED DESCRIPTION

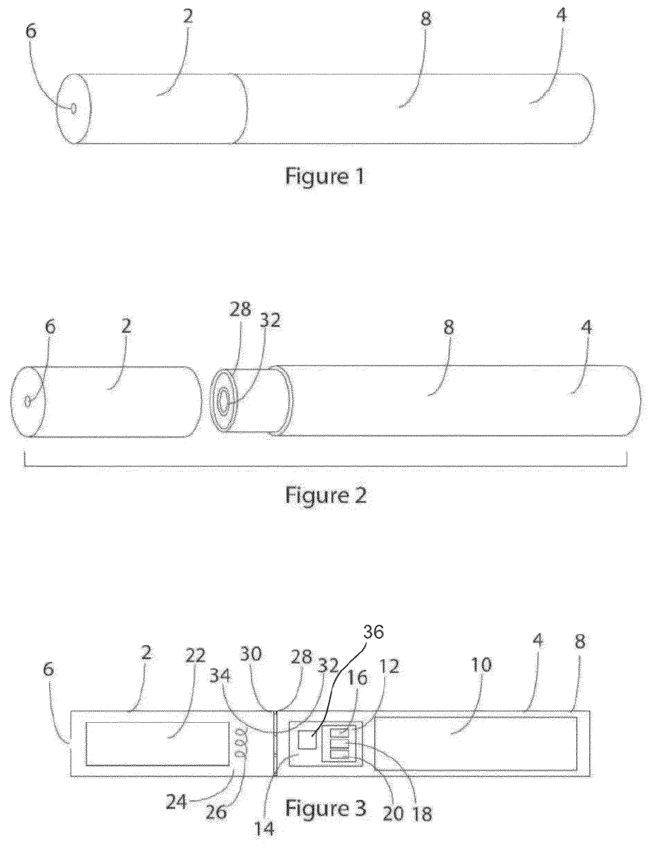

Referring to FIGS. 1 to 3 there is shown an electronic vapor provision device, also herein referred to as an electronic smoking device, that comprises a mouthpiece 2 and a body 4. The electronic vapor provision device is shaped like a conventional cigarette, both the mouthpiece 2 and body 4 are cylindrical and are configured to connect to each other coaxially so as to form the conventional cigarette shape. The mouthpiece 2 is connectable to the body 4 at a first end of the mouthpiece 2 and has an air outlet 6 at a second end. The body 2 comprises a battery assembly 8, comprising a power cell 10 and a computer 12 on a circuit board 14, wherein the power cell 10 is connected to the computer 12. The computer 12 comprises a computer processor 16, a memory 18 and input-output arrangement 20. In this example the computer 12 is a microcontroller. The computer 12 is configured to control and interface with the other electrical components of the battery assembly 8, comprising the power cell 10, via the input-output arrangement 20.

The mouthpiece 2 comprises a liquid bottle 22 and a vaporizer 24 having a heater coil 26. For example, the vaporizer 24 is in fluid communication with the liquid bottle 22. The mouthpiece 2 is connectable to the battery assembly 8 by a screw thread, wherein connection of the battery assembly 8 and the mouthpiece 2 connects a first battery assembly terminal 28 to a first vaporizer terminal 30 and a second battery assembly terminal 32 to a second vaporizer terminal 34, forming an electrically conductive contact in both cases. The vaporizer terminals 30 34 are electrically connected in parallel to the heater coil 26 of the vaporizer 24.

The herein described configuration of the computer 12 comprises the computer operating according to a computer program stored in its memory 18 and accessed by its computer processor 16.

The device is configured such that, if the computer determines that the vaporizer 24 is connected to the battery assembly 8, then the computer 12 initially remains in a normal operation mode.

Moreover, to maximize the lifetime of the charge in the power cell 10, the device is configured such that, if the computer 12 determines that the vaporizer 24 is not connected to the battery assembly 8, then the computer 12 enters a low power sleep mode.

Furthermore, to maximize the lifetime of the charge in the power cell 10 while the computer 12 determines that the vaporizer 24 is connected, the computer 12 is configured to detect when the device has been inactive for a predetermined inactive time such as 12 minutes. This inactive time is the time that has elapsed since use of the device by the user. Moreover, in response to detecting that the device has been inactive for the inactive time, the computer 12 is configured to leave normal operational mode and to enter a low power sleep mode. For example, sleep mode may comprise the computer 12 consuming minimal power and performing no processing. A period during which the computer 12 is in sleep mode is herein referred to as sleep time. Furthermore, the device is configured such that, in order to return the device to a normal operational mode, a user is required to disconnect the vaporizer 24 from the battery assembly 8 and then reconnect the vaporizer 24 to the battery assembly 8. This configuration of the device comprises the computer 12 being configured to detect when the vaporizer 24 has been disconnected and then reconnected to the battery assembly 8. Moreover, in response to detection by the computer 12 of disconnection and reconnection of the vaporizer 24, the computer 12 is configured to leave sleep mode and re-enter the normal operation mode.

The computer 12 determining whether the vaporizer 24 is connected to the battery assembly 8 and/or detecting whether the vaporizer 24 has been disconnected and then reconnected may for example comprise the computer 12 periodically checking whether the vaporizer 24 is connected to the battery assembly 8 during both normal operation mode and sleep mode.

For example, after determining that the vaporizer 24 is not attached and entering sleep mode, the computer 12 may then remain in sleep mode for an initial sleep time of two seconds. After the sleep time, the computer 12 wakes and immediately and quickly checks again for a vaporizer connection. Again, if the vaporizer 24 is not connected the computer 12 goes into sleep mode for another two seconds. The time that the computer 12 is awake is extremely short compared to the sleep time so the circuit remains predominantly in a low power mode, thus conserving power. During the sleep time no checks are made to determine whether the vaporizer 24 is attached however a user may take several seconds to assembly the device, connecting the vaporizer 24 and the battery assembly 8, so the vaporizer 24 connection may be easily established by the computer 12 before use of the device by a user.

Furthermore, after determining that the vaporizer 24 is connected and that the device has been inactive in this connected state for the inactive time and then entering sleep mode, the computer 12 may then remain in sleep mode for an initial sleep time of two seconds. After the sleep time, the computer 12 wakes and immediately and quickly checks again for a vaporizer connection. Again, if the vaporizer is still connected the computer 12 goes into sleep mode for another two seconds. The time that the computer 12 is awake is extremely short compared to the sleep time so the circuit remains predominantly in a low power mode, thus conserving power. During the sleep time no checks are made to determine whether the vaporizer 24 is attached. However a user may take several seconds to disconnect the vaporizer 24 and several seconds to then reconnect the vaporizer 24, so the computer 12 may easily detect and log the disconnection and subsequent reconnection. In this way the computer 12 is able to detect the disconnection and subsequent reconnection of the vaporizer 24 by a user and to respond accordingly by leaving sleep mode and entering normal operation mode.

The waking of the computer 12 may for example comprise the computer 12 entering a waking mode distinct from the sleep mode and the normal operation mode.

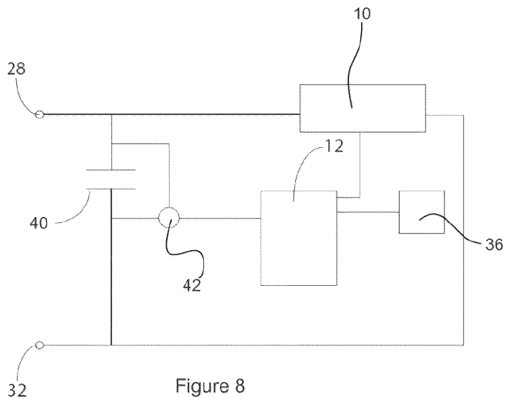

The computer 12 checking whether the vaporizer 24 is connected to the battery assembly 8 may for example comprise the computer 12 sending an electrical pulse to the battery assembly terminals 28, 32. For example, the computer 12 may control the power cell 10 so as to supply a pulse of current to the first battery assembly terminal 28 and may measure the current reaching the second the battery assembly terminal 32, for example using a digital multimeter of the battery assembly 8. The digital multimeter is referenced 44 in the circuit diagram of FIG. 8. Furthermore, if the vaporizer 24 is connected, the digital multimeter reads a current reaching the second battery assembly terminal 32 via the vaporizer 24 and provides information to the computer 12 indicating this.

The battery assembly 8 of the electronic cigarettes described herein further comprises an air pressure sensor 36, wherein the air pressure sensor is powered by the power cell 10 and controlled by the computer 12. Once the vaporizer is connected to the battery assembly 8, and while the device is still in the normal operation mode resulting from the processors detection of this connection, in order to use the device the user must suck on the mouthpiece 2. The electronic cigarette is configured such that the user sucking on the mouthpiece 2 causes a drop in air pressure at the air pressure sensor 36. The computer 12 therefore receives information from the air pressure sensor 36 indicating that a user is sucking on the device. In response to this information, the computer 12 controls the power cell 10 to power the vaporizer 24. For example, the computer may control the power cell 10 to power the vaporizer 24 via the respective first and second terminals of both the battery assembly and the vaporizer. This causes the vaporization of liquid communicated to the vaporizer 24 from the liquid bottle 22. Consequently, use of the device by a user comprises the user sucking on the device and the detection of this user interaction by the device so as to trigger the vaporization of the liquid contained in the device. The provided vapor then passes to the user. The vapor provision device is configured such that a user can only use the device, by sucking on the mouthpiece 2, when the computer is in the normal operation mode. The normal operation mode can therefore be described as a usable mode.

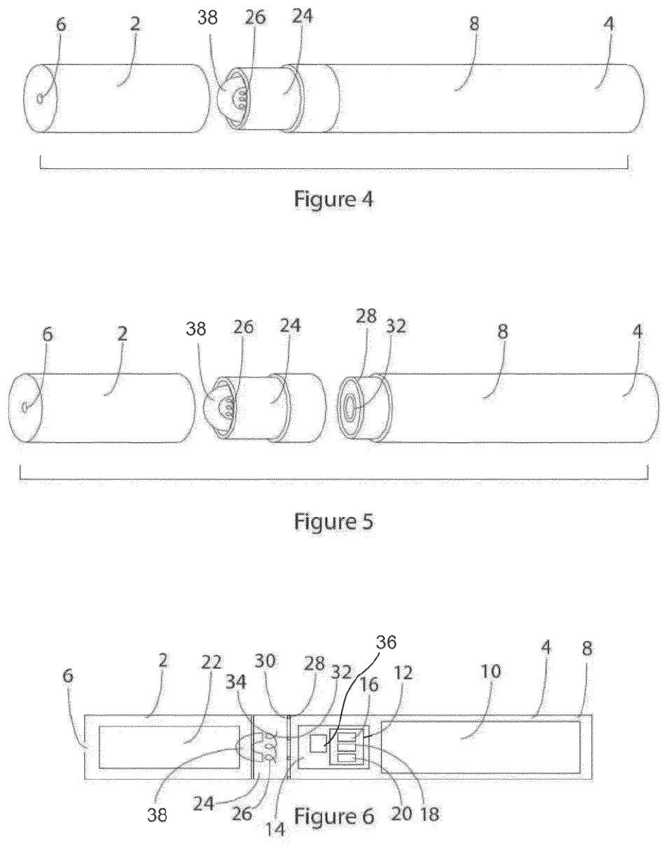

FIGS. 4 to 6 show another example of an electronic vapor provision device. This device is similar to that shown in FIGS. 1 to 3. However in this example the vaporizer 24 does not form part of the mouthpiece 2. The mouthpiece 2 contains a liquid bottle 22 and is attachable to the vaporizer 24. The vaporizer 24 has a heater coil 26 and additionally a wick 38. For example the wick 38 may be a mesh wick. The mouthpiece 2 and the vaporizer 24 are configured to connect to each other such that the wick 38 acts to communicate liquid from the liquid container 22 onto the vaporizer 24. The configuration of the device involving the conservation of power based on interaction between the vaporizer 24 and the battery assembly 8 involving the disconnection and reconnection of the vaporizer is as described above.

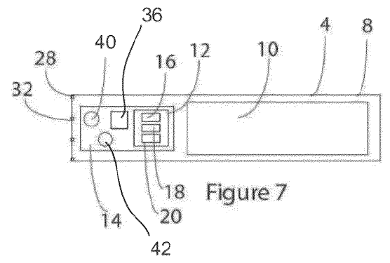

A further example of how, in the cigarettes of FIGS. 1 to 6, connection of the vaporizer 24 to the body may be detected by the computer 12 is now described with reference to FIGS. 7 and 8. With respect to FIGS. 7 and 8, the battery assembly 8 of the electronic vapor provision device is similar to that shown in FIG. 1 to FIG. 6, additionally comprising a capacitor 40 and a digital multimeter 42. The capacitor 38 is arranged in a circuit such that it is in parallel to the battery assembly terminals and to the power cell 10. The digital multimeter 42 of the battery assembly 8 is wired in a switched parallel circuit to the capacitor 40. To test whether the vaporizer 24 is connected to the battery assembly 8, the computer 12 first controls the power cell to charge the capacitor 40, then waits a short time and checks the charge of the capacitor 40. The computer 12 checks the charge of the capacitor 40 by triggering the completion of the switched digital multimeter circuit and then receives information from the multimeter 42 indicating a voltage across the capacitor 40 resulting from the charge of the capacitor 40. If the vaporizer 24 is connected, the resistance of the vaporizer 24 causes the capacitor 40 to discharge quickly so the computer 12 measures at least a substantially fully discharged capacitor 40. If the vaporizer 24 is not connected the capacitor 40 is not substantially fully discharged when checked by the computer 12.

With regard to the embodiments described herein, the following alternatives and variations will now be described.

The electronic vapor provision devices described may be electronic cigarettes. However, the device is not restricted to being cigarette shaped.

The sleep time may be substantially 2 seconds. However, the sleep time is not restricted to 2 seconds and other suitable values could be used. Moreover, the time between entering sleep modes can be significantly less than the sleep time.

The computer processor 16 can be a microprocessor. Moreover, the computer 12 may comprise a microcontroller. Furthermore, a computer such as a microcontroller could utilize a watchdog timer to implement the sleep time wait in the low power mode. Using a microcontroller has space saving advantages since the entire computer is located on a single chip and therefore the size of the device is minimized. Fewer components to assemble also provides reduced manufacturing times are costs. The computer is not restricted being a microcontroller and could be fabricated from separate processor, memory and input-output components.

The vaporizers 24 described are examples only.

Moreover, the sleep mode may be the lowest non-zero power mode of the device.

Although an air pressure sensor 36 is described, other configurations may be employed to detect when a user is attempting to use the device. For example, an airflow sensor may be used and the device may be configured such that sucking on the mouthpiece 2 by a user causes a flow of air past the airflow sensor.

Although a liquid bottle 22 is described, other types of liquid storage may be used. For example the device may comprise foam partially saturated in liquid for vaporization.

Although a digital multimeter 42 is described as being used by the computer 12 to determine the level of charge of the capacitor 40, other suitable configurations may be employed for this purpose. For example, a digital voltmeter may instead be used.

The electronic vapor provision devices are described as being configured such that the computer 12 can determine when the vaporizer 24 is connected to the battery assembly 8 of the body 4. This configuration of the electronic vapor provision device may be described as the device comprising a sensor arrangement configured to detect when the vaporizer is connected and to provide information to the processor accordingly. For example, with regard to the device of FIGS. 7 and 8, the first and second battery assembly terminals 28 32, the capacitor 40 and the digital multimeter 42 may together be considered as a sensor arrangement of the device. In this sense, the sensor arrangement may take other forms to that described. For example, the sensor arrangement may comprise only one sensor, for instance a pressure sensor configured to detect a mechanical pressure resulting from connection of the vaporizer 24 to the body 4.

The various embodiments described above facilitate a number of improvements.

The described configuration of the electronic vapor provision device is such that in the case of the vaporizer 24 being connected and the device entering the sleep mode due to the device being inactive for the inactive time, the device can not then accidentally enter a normal mode. Moreover, a user must be aware of the requirement to disconnect and then reconnect the vaporizer 24 and must carry this procedure out in order to trigger the device to enter the usable, normal operation mode. Entering a normal mode requires effort from the user. This facilitates the advantage of increased safety and control of use of the device.

Described operational modes and configurations of the computer may be operational modes and configurations of the processor.

Although examples have been shown and described, it will be appreciated by those skilled in the art that various changes and modifications might be made without departing from the scope of the invention.

In order to address various issues and advance the art, the entirety of this disclosure shows by way of illustration various embodiments in which the claimed invention(s) may be practiced and provide for superior electronic vapor provision devices. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future. Any feature of any embodiment can be used independently of, or in combination with, any other feature.

1. An electronic vapor provision device comprising a body and a vaporizer, wherein
the body comprises a power cell and a computer, the computer comprising a processor, a memory, and an input-output means;
the vaporizer is releasably connectable to the body;
the computer is configured to leave a normal operation mode and enter a low power sleep mode when the vaporizer is connected to the body and the device is inactive for an inactive time, wherein the device is configured such that a user can only use the device when the computer is in the normal operation mode; and
the computer is configured to leave the sleep mode and enter the normal operation mode,
wherein the device is configured such that the device is unable to accidentally enter the normal operation, such that to enter the normal operation mode the user is required to exert effort.

2. The electronic vapor provision device of claim 1, wherein the electronic vapor provision device is an electronic cigarette.

3. The electronic vapor provision device of claim 1, wherein the sleep mode is the lowest non-zero power mode of the device.

4. The electronic vapor provision device of claim 1, wherein the electronic vapor provision device uses less power in sleep mode than in usable mode.

5. The electronic vapor provision device of claim 1, wherein the electronic vapor provision device is inactive when not being used as a vapor provision device by a user.

6. The electronic vapor provision device of claim 1, wherein
the body comprises first and second body connection terminals, and the vaporizer comprises first and second vaporizer connection terminals;
the device is configured such that when the vaporizer is connected to the body, the first body connection terminal is connected to the first vaporizer connection terminal and the second body connection terminal is connected to the second vaporizer connection terminal.

7. The electronic vapor provision device of claim 1, wherein
the body further comprises a capacitor; and
the processor is configured to first charge the capacitor and then detect whether the vaporizer is connected to the body by determining whether the capacitor is discharged.

8. The electronic vapor provision device of claim 7, wherein the capacitor is connected in parallel with the first and second body connection terminals.

9. The electronic vapor provision device of claim 7, wherein the computer is configured to send out a pulse and the capacitor is charged for a period of time equal to the width of the pulse.

10. The electronic vapor provision device of claim 9, wherein the pulse is a square wave.

11. The electronic vapor provision device of claim 1, wherein the vaporizer further comprises a mesh wick.

12. The vaporizer of the electronic vapor provision device of claim 1.

13. An electronic vapor provision device body, comprising a power cell and a computer, the computer comprising a processor, a memory, and an input-output means, and the electronic vapor provision device body configured to be releasably connectable to a vaporizer; wherein
the computer is configured to leave a normal operation mode and enter a low power sleep mode when the vaporizer is connected to the electronic vapor provision device body and the electronic vapor provision device body is inactive for an inactive time, wherein the electronic vapor provision device body is configured such that a user can only use the electronic vapor provision device body when the computer is in the normal operation mode; and
the computer is configured to leave the sleep mode and enter the normal operation mode, wherein the electronic vapor provision device body is configured such that the electronic vapor provision device body is unable to accidentally enter the normal operation, such that to enter the normal operation mode the user is required to exert effort.

14. A method of triggering a change in the mode of a processor of an electronic vapor provision device from a low power sleep mode to a normal operation mode, wherein
the electronic vapor provision device comprises a body and a vaporizer;
the body comprises a power cell and a computer, the computer comprising the processor, a memory, and an input-output means; and
the vaporizer is releasably connectable to the body;
the method comprising leaving a normal operation mode and entering a low power sleep mode when the vaporizer is connected to the body and the device is inactive for an inactive time, wherein the device is configured such that a user can only use the device when the computer is in the normal operation mode;
leaving the sleep mode and entering the normal operation mode, wherein the device is configured such that it is unable to accidentally enter the normal operation, such that to enter the normal operation mode the user is required to exert effort.
